(19) United States
(12) Patent Application Publication
(10) Pub. No.: US 2005/0049663 A1
(43) Pub. Date: Mar. 3, 2005

(54) Percutaneous flat lead introducer

(76) Inventors: Charmaine K. Harris, Woodbury, MN (US); Joseph J. Klein, Plymouth, MN (US)

Correspondence Address: SHUMAKER & SIEFFERT, P. A., 8425 SEASONS PARKWAY, SUITE 105, ST. PAUL, MN, 55125, US

(21) Appl. No.: 10/718,038

(22) Filed: Nov. 20, 2003

Related U.S. Application Data
(60) Provisional application No. 60/499,207, filed on Aug. 29, 2003.

(51) Int. Cl. A61N 1/05

(52) U.S. Cl. 607/115

(57) ABSTRACT

In general, the invention is directed to a technique for percutaneously introducing a stimulation lead into a target stimulation site via the epidural region proximate the spine of a patient. The process of introducing the stimulation lead may include the use of a hollow stimulation lead introducer, which comprises an elongated sheath and an elongated dilator. The dilator fits within the sheath and serves to widen a path through the epidural region for the introduction of a stimulation lead. At least a portion of the stimulation lead introducer has an oblong cross-section, allowing passage of stimulation leads such as paddle leads. The stimulation lead introducer may enter the epidural region proximate a spine of a patient via a guidewire. The stimulation lead introducer provides a path through the epidural region of a patient to a target stimulation site. A stimulation lead may travel through the path to reach the target stimulation site where it may provide therapy to the patient.

### TECHNICAL FIELD

This application claims priority from U.S. provisional application Ser. No. 60/499,207, filed Aug. 29, 2003, the entire content of which is incorporated herein by reference.

### TECHNICAL FIELD

The invention relates to neurostimulation systems and, more particularly, to stimulation lead introducers.

### BACKGROUND

Neurostimulation systems may be used to deliver neurostimulation therapy to patients to treat a variety of symptoms or conditions such as chronic pain, tremor, Parkinson's disease, multiple sclerosis, spinal cord injury, cerebral palsy, amyotrophic lateral sclerosis, dystonia, torticollis, epilepsy, incontinence, or gastroparesis. A neurostimulation system delivers neurostimulation therapy in the form of electrical pulses. In general, neurostimulation systems deliver neurostimulation therapy via electrodes on stimulation leads located proximate to the spinal cord, pelvic nerves, or stomach, or within the brain of a patient.

The stimulation leads may include percutaneously implanted leads or surgically implanted leads. Surgically implanted leads are often larger and wider than traditional percutaneously implanted leads. For example, surgically implanted leads may include paddle-shaped leads with surface electrodes. Surgically implanted leads are often desirable because they are less susceptible to migration, include unidirectional electrode arrays, and provide reduced power consumption. Although surgical leads can provide more effective leads, percutaneously implanted leads are often preferred because they are implanted in a less invasive manner.

### SUMMARY

In general, the invention is directed to techniques for percutaneously introducing a generally flat stimulation lead into a target stimulation site via the epidural region proximate the spine of a patient. A number of electrodes on the stimulation lead, which may be a paddle-like lead, rest at a target stimulation site where the electrodes can provide stimulation therapy to the patient.

The process of introducing the stimulation lead includes the use of a hollow stimulation lead introducer, which comprises an elongated sheath and an elongated dilator. The dilator fits within the sheath and serves to widen a path through the epidural region for the introduction of the sheath, and ultimately the stimulation lead. At least a portion of the stimulation lead introducer has an oblong cross-section, allowing passage of flat stimulation leads such as paddle leads.

The stimulation lead introducer may enter the epidural region proximate the spine of a patient via a guidewire. The stimulation lead introducer provides a path through the epidural region of a patient to a target stimulation site. The stimulation lead travels along the path defined by the lead introducer to reach the target stimulation site where it is positioned to deliver therapy to the patient.

In one embodiment, the invention is directed to a stimulation lead introducer comprising an elongated dilator defining a dilator lumen sized to advance over a guidewire, the dilator having a substantially conical distal tip, wherein at least a portion of the conical distal tip has a substantially oblong cross-section, and an elongated sheath defining a sheath lumen sized to accommodate the dilator or the stimulation lead.

In another embodiment, the invention is directed to a method for introducing a stimulation lead comprising inserting a stimulation lead introducer into an epidural region proximate a spine of a patient via a guidewire, wherein the introducer includes an elongated dilator defining a dilator lumen sized to advance over the guidewire, the dilator having a substantially conical distal tip, wherein at least a portion of the conical distal tip has a substantially oblong cross-section, and an elongated sheath defining a sheath lumen sized to accommodate the dilator or the stimulation lead, withdrawing the dilator from the sheath, and introducing a stimulation lead to a target site within the epidural region via the sheath.

In a further embodiment, the invention is directed to a dilator for widening a path for a stimulation lead to travel through an epidural region proximate a spine of a patient, the dilator having a proximal end and a distal end, wherein the dilator defines a dilator lumen sized to advance over a guidewire, the dilator having a substantially conical distal tip, wherein at least a portion of the conical distal tip has a substantially oblong cross-section.

The invention may provide one or more advantages. For example, the invention permits percutaneous introduction of leads that ordinarily require surgical implantation. In particular, generally flat, or “paddle-like,” leads may be introduced into the epidural region proximate a spine of a patient without the need for surgical intervention. Instead, the dilator and sheath associated with the invention permit introduction of flat leads by less invasive, percutaneous incision, reducing patient trauma and recovery time.

The invention may further provide more customizable components for introducing the stimulation lead. In particular, at least one of the sheath and the dilator may include deformable material, such as polyethylene. The deformable properties of the material allow the dilator to be formed to fit the anatomy of a patient more precisely. In addition, the deformable properties of the stimulation lead introducer may reduce the possibility of causing a “wet tap”, or cerebral spinal fluid (CSF) leak. A CSF leak may cause severe headaches or, if the leak is severe, neurological damage.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a diagram illustrating a stimulation lead introducing kit, which includes components for percutaneously implanting a stimulation lead.

FIG. 2 is a perspective view of a sheath and dilator useful in the stimulation lead introducing kit of FIG. 1.

FIG. 3 is a perspective diagram illustrating an exemplary dilator that may be used for percutaneously implanting a stimulation lead.

FIG. 4 is a perspective diagram illustrating an exemplary sheath that may be used for percutaneously implanting a stimulation lead.

FIG. 5 is a perspective diagram illustrating a stimulation lead introducer, including a dilator and elongated sheath that may be used for percutaneously implanting a stimulation lead.

FIG. 6 is a cross-sectional diagram of a stimulation lead introducer, including a dilator and an elongated sheath that may be used for percutaneously implanting a stimulation lead.

FIG. 7 is a cross-sectional diagram of a stimulation lead passed through a sheath.

FIG. 8 is a flow diagram illustrating an exemplary technique for percutaneously implanting a stimulation lead by using a stimulation lead introducer.

### DETAILED DESCRIPTION

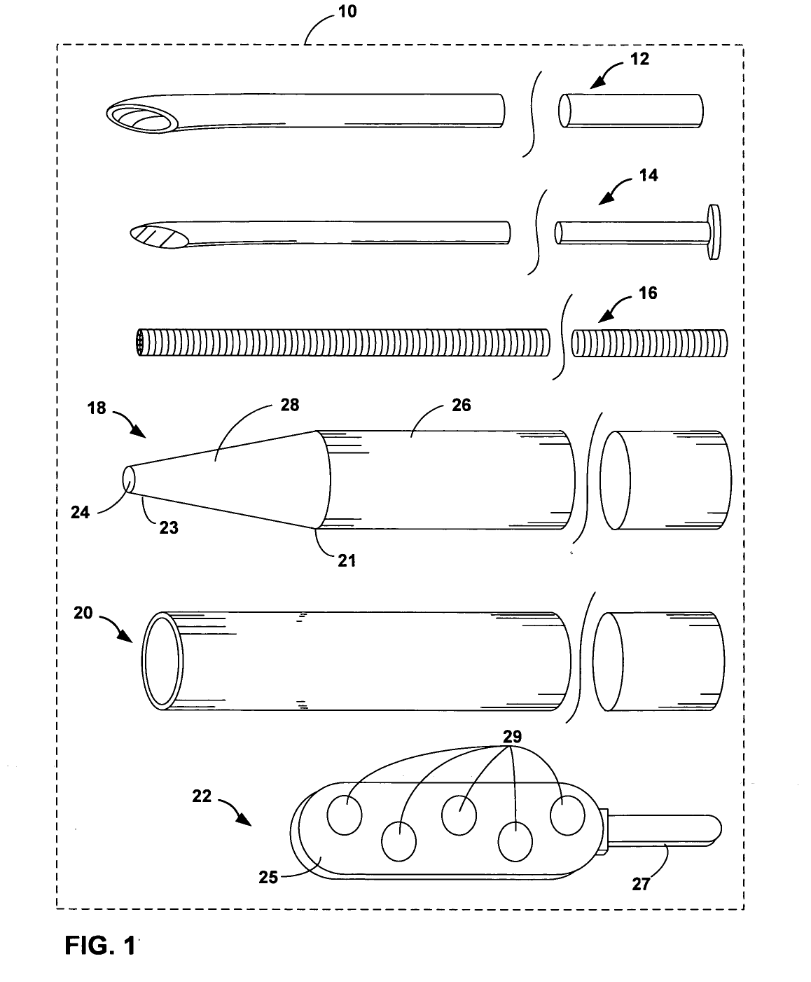

FIG. 1 is a diagram illustrating a stimulation lead introducing kit 10, which includes components for percutaneously implanting a generally flat stimulation lead. In particular, with kit 10, a generally flat, or “paddle-like,” stimulation lead may be percutaneously implanted through the epidural region proximate a spine of a patient. In this manner, surgical implantation procedure can be avoided. As shown in FIG. 1, kit 10 includes a needle 12, a stylet 14, a guidewire 16, a dilator 18, a sheath 20, and a stimulation lead 22. The diagram of FIG. 1 depicts the distal ends and proximal ends of the parts in kit 10 at the left and right, respectively. In general, a “distal” end will refer to the first end of a component that is introduced into the patient, whereas the “proximal” generally extends outside of the body for manipulation by medical personnel.

Needle 12 has a lumen that may be between 14 gauge and 18 gauge to allow needle 12 to receive stylet 14. In some instances, needle 12 may take the form of a modified Tuohy needle, which has an opening that is angled 45 degrees so that an instrument passing through the needle exits through the needle at an angle. Stylet 14 is sized to fit inside needle 12. Stylet 14 fills the lumen of needle 12 to prevent coring in the tissue of a patient when needle 12 is inserted into the patient.

Guidewire 16 is an elongated, flexible instrument with a cross-sectional width sized to fit within needle 12 upon withdrawal of stylet 14. In some embodiments, guidewire 16 may have an outer diameter of approximately 0.05 inches. Guidewire 16 is generally cylindrical, and may be steerable to permit deployment of the guidewire to a desired “target” site within the epidural region. In practice, guidewire 16 may be inserted through needle 12 and steered through the epidural region to the target site for neurostimulation therapy.

Guidewire 16 prepares a path for other medical instruments in kit 10 to traverse. In particular, guidewire 16 provides a path that is clear of obstructions so a stimulation lead introducer, formed by dilator 18 and sheath 20, can reach the target site by advancing over guidewire 16.

Dilator 18 is an elongated, hollow structure that has a dilator proximal end and a dilator distal end. Dilator 18 defines a dilator body 26 with a dilator lumen extending between the dilator proximal end and dilator distal end and sized to advancing over guidewire 16. A substantially conical distal tip 28 extends from the distal end of dilator body 18. The conical tip, comprising a proximal portion 21 and distal portion 23, widens a path through the epidural region for dilator body 26 to pass through. At least a portion of the dilator 18 has a substantially oblong cross-section, which allows a flat, paddle-like stimulation lead 22 to fit through the widened path.

In one embodiment, the proximal portion 21 of the conical structure 28 has a substantially oblong cross-section, whereas the distal portion 23 has a generally circular cross-section. An opening 24 is provided at distal portion 23. Hence, substantially conical distal tip 28 may taper from an oblong cross-section at proximal portion 21 to a circular cross-section at distal portion 23, and hence a circular opening 24. In addition, the dilator lumen may have a substantially oblong cross-section.

Dilator 18 may be made of an extruded or molded material. The material may include a substantially deformable material, such as polyethylene. The deformable properties of the material allow dilator 18 to be formed to fit the anatomy of a patient more accurately. In some cases, a physician may be able to shape or form a portion of the dilator 18, such as distal tip 28, to a desired configuration. To that end, the material in distal tip 28 may be selected to hold a shape. In addition, the deformable properties of the stimulation lead introducer decrease the probability of causing a “wet tap”, or CSF leak, which is an event that may cause severe headaches or, if the leak is severe, may cause neurological damage. A CSF leak may occur if the stimulation lead introducer is inserted too far into the epidural region, causing a puncture in the dura membrane of the epidural region. The deformable properties of the stimulation lead introducer may allow the distal tip 28 to deform enough to prevent a CSF leak. In particular, distal tip 28 may bend to the side upon reaching the dura membrane, preventing a puncture in the dura membrane.

Sheath 20, an elongated, hollow structure defining an inner sheath lumen, includes a sheath proximal end and a sheath distal end. In one embodiment, the outside of sheath 20 has a substantially oblong cross-section. In another embodiment the sheath lumen has a substantially oblong cross-section. Thus, in some embodiments, the entire sheath 20 has an oblong cross-section, while in other embodiments, the sheath outer diameter has a non-oblong cross-section and the inner lumen has an oblong cross-section. The term “oblong,” with respect to dilator 18 and sheath 20, refers generally to a cross-section, taken normal to the longitudinal extent of the respective component, with a width that is substantially greater than its height.

Like dilator 18, sheath 20 may be made of extruded or molded material. The material may include a substantially deformable material, such as polyethylene. The deformable properties of the material allow sheath 20 to be formed to fit the anatomy of a patient more accurately. In addition, sheath 20 may include radio-opaque material that is viewable under fluoroscopic imaging to aid medical personnel in visualizing the sheath during percutaneous introduction.

In practice, sheath 20 fits over dilator 18 to form the stimulation lead introducer. In addition, sheath 20 allows for the passage of a stimulation lead when dilator 16 is not present in sheath 20, i.e., upon withdrawal of dilator 16. In one embodiment, sheath 20 may be shorter than dilator 18 so that the conical distal tip 28 of dilator 18 extends beyond the distal end of sheath 20.

Stimulation lead 22 may include a paddle-shaped, flat structure with at least one electrode 29 to provide stimulation to a patient, as shown in FIG. 1. FIG. 1 generally depicts a distal end of stimulation lead, including electrode surface 25 and lead body 27. In operation, proximal end of lead body 27 is coupled to a neurostimulator that generates neurostimulation energy for delivery via electrodes 29. Stimulation lead 22 is shown in FIG. 1 with five surface electrodes 29. In various embodiments, the stimulation lead is a paddle lead. For example, the stimulation lead may take the form of a quad-electrode paddle lead, an octet-electrode paddle lead, and a deployable paddle lead. A line of neurostimulation paddle leads are commercially available from Medtronic, Inc. of Minneapolis, Minn.

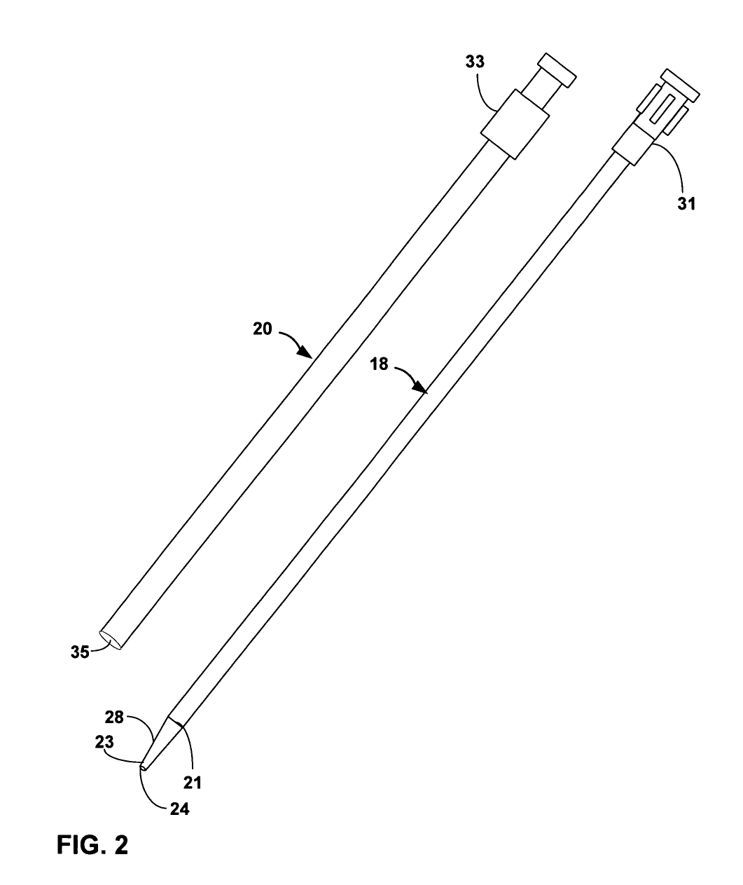

FIG. 2 is a perspective view of sheath 20 and dilator 18 in stimulation lead introducing kit 10 of FIG. 1. As shown in FIG. 2, a proximal ends of sheath 20 and dilator 18 may include respective fittings 31, 33 to accommodate insertion of dilator 18 into sheath 20. Dilator 18 may be sized longer than sheath 20, so that distal tip 28 protrudes through an opening 35 in a distal portion of sheath 20.

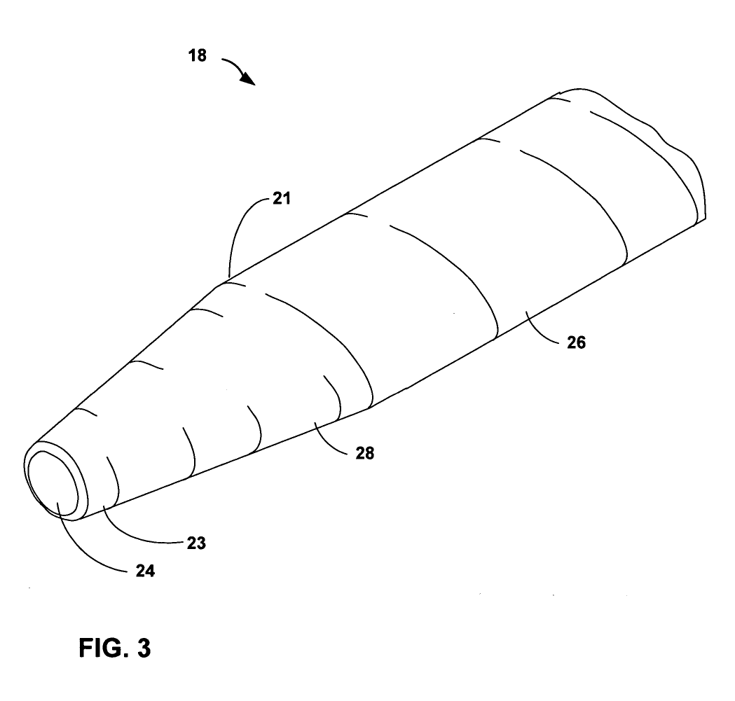

FIG. 3 is a perspective diagram illustrating an exemplary dilator 18 that may be used for percutaneously implanting a stimulation lead 22. In particular, dilator 18 is part of a stimulation lead introducer that also includes sheath 20. Dilator 18 is an elongated, hollow structure that has a dilator proximal end and a dilator distal end. Dilator 18 defines a dilator body 26 with a dilator lumen extending between the dilator proximal end and dilator distal end for advancing over guidewire 16. A substantially conical distal tip 28 extends from the distal end of dilator 18, which has an opening 24. As shown in FIG. 3, opening 24 may be substantially circular to fit around guidewire 16. Alternatively, opening 24 may have a non-circular cross-section. For example, opening 24 may have an oblong cross-section.

The conical tip 28 of dilator 18, comprising a proximal and distal end, serves to widen a path through the epidural region for the rest of the stimulation lead introducer to pass through. At least a portion of dilator 18 may have an oblong cross-section so a flat, paddle-like stimulation lead 22 can fit in the widened path. In a further embodiment, the dilator lumen has a substantially oblong cross-section. As shown in FIG. 3, the proximal portion 21 of the conical tip 28, adjacent dilator body 26, may have an oblong cross-section. Also shown in FIG. 3, the body of dilator 18 has an oblong cross-section.

In one embodiment, the proximal opening may have a width of approximately 0.175 inches, and the distal opening 24 may have a width of approximately 0.05 inches. Hence, the proximal width may be greater than approximately three times the distal width. In one embodiment, the dilator body 26 has a height that is greater than the height of distal opening 24. The height and width of dilator 18 may be designed to fit a particular stimulation lead that may be passed through the path created by needle 12 and dilator 18. In one embodiment, dilator 18 may be longer than sheath 20 so the conical structure 28 of dilator 18 extends past the distal end of sheath 20.

Dilator 18 may comprise extruded or molded material. The material may include a deformable material such as a polymer. In particular, the polymer may comprise polyethylene. The deformable properties of the material allow dilator 18 to be formed to better fit the anatomy of a patient.

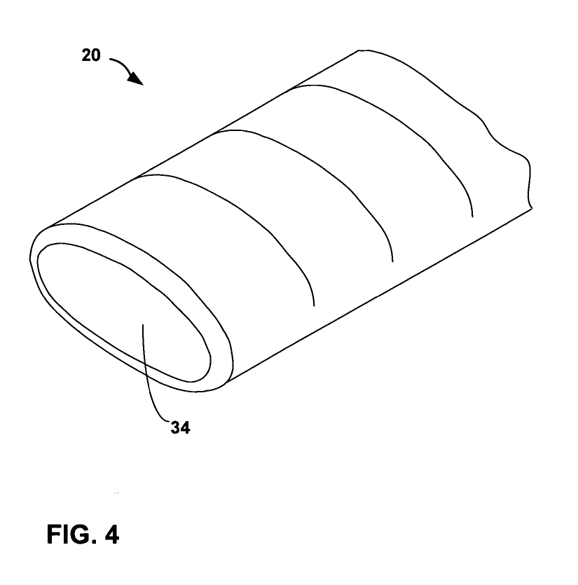

FIG. 4 is a perspective diagram illustrating a distal portion of an exemplary sheath 20 that may be used for percutaneously implanting a stimulation lead. Sheath 20, an elongated, hollow structure defining a sheath lumen, includes a sheath proximal end and sheath distal end. In one embodiment, the outside of sheath 20 has a substantially oblong cross-section. In another embodiment the sheath lumen has a substantially oblong cross-section so stimulation lead 22 can fit in the widened path. As shown in FIG. 4, the sheath lumen may have a substantially oblong cross-section. In one embodiment, the proximal opening 34 may have a width of approximately 0.175 inches, and a height of approximately 0.05 inches. The height and width of sheath 20 may be designed to fit a particular stimulation lead that may be passed through the path created by a needle 12, a guide wire 16, and a dilator 18.

Sheath 20 may comprise extruded or molded material. The material may include a deformable material such as a polymer. In particular, the polymer may comprise polyethylene. The deformable properties of the material allow dilator 18 to be formed to better fit the anatomy of a patient. In addition, sheath 20 may include radio-opaque material that is viewable under fluoroscopic imaging.

In practice, sheath 20 is part of a stimulation lead introducer that also includes dilator 18. In one embodiment, sheath 20 may be shorter than dilator 18. A substantially conical structure 28 on the distal end of dilator 18 may extend past the distal end of sheath 20. In addition, sheath lumen of sheath 20 allows for the passage of a stimulation lead when dilator 16 is not present within sheath 20.

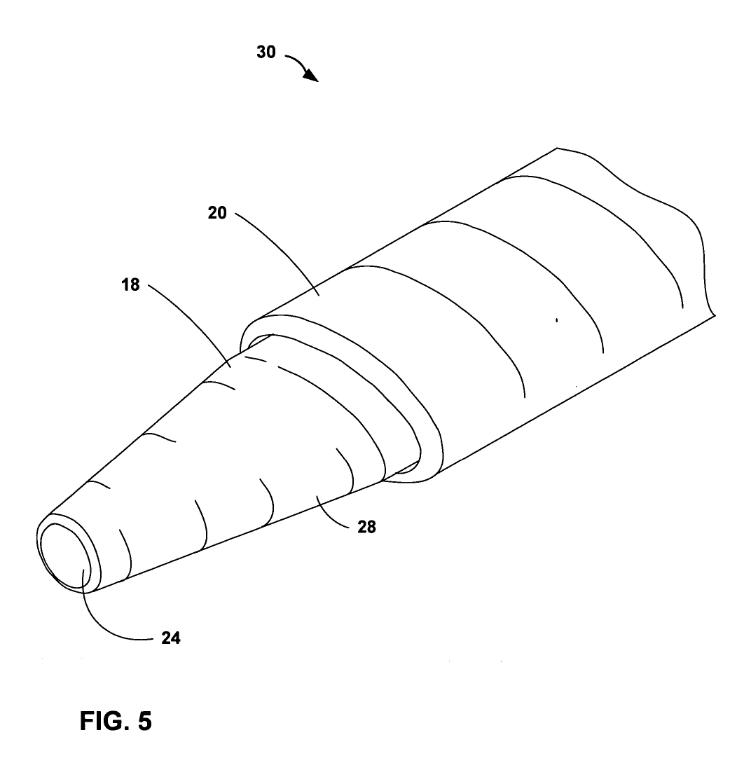

FIG. 5 is a perspective diagram illustrating a stimulation lead introducer 30, which may be used for percutaneously implanting a stimulation lead. In particular, stimulation lead introducer 30 includes elongated sheath 20, which may fit over dilator body 26 of dilator 18. More particularly, dilator 18 may fit within a sheath lumen defined by sheath 20. Dilator 18, which comprises a dilator proximal end and a dilator distal end, has a substantially conical structure 28 extending from the dilator distal end. The distal end of the conical structure 28 has an opening 24, which may be sized to advance over guidewire 16. In one embodiment, dilator 18 is at least as long as sheath 20 so the conical structure 28 of dilator 18 extends through the distal end of sheath 20.

At least a portion of the stimulation lead introducer has a substantially oblong cross-section. As shown in FIG. 5, the proximal end of the conical structure 28 at a distal end of dilator 18 may have an oblong cross-section, while the opening 24 may have a circular cross-section. Hence, distal tip 28 is generally conical but may have different cross-sectional shapes at the proximal and distal ends of the distal tip. Also shown in FIG. 5, sheath 20 of stimulation lead introducer 30 may have a substantially oblong cross-section. In addition, the sheath lumen may have a substantially oblong cross-section.

At least a portion of the stimulation lead introducer 30 may include deformable material. In particular, sheath 20 or dilator 18 may include a deformable material such as polyethylene. The deformable properties of the material allow sheath 20 to be formed to fit the anatomy of a patient more accurately. In addition, at least a portion of stimulation lead introducer 30 may include a radiopaque material that is viewable under fluoroscopic imaging. In particular, sheath 20 may include the radiopaque material.

In practice, stimulation lead introducer 30 enters the epidural region of a patient by advancing over the guidewire 16, which prepares a path to a target site for stimulation lead introducer 30 to follow. An imaging technique may aid the introduction of the stimulation lead introducer 30. For example, as discussed above, fluoroscopic imaging may be used to follow the progress of stimulation lead introducer 30 as it advances over guidewire 16. The stimulation lead introducer 30 may widen the path to the target site. In particular, stimulation lead introducer 30 may widen the path so that the cross-section of the path is substantially oblong.

After stimulation lead introducer 30 widens the path for stimulation lead 22 to get to the target site, dilator 18 may be withdrawn. Sheath 20 remains in the epidural region, maintaining the path for the introduction of stimulation lead 22. A stimulation lead 22 may be introduced via sheath 20 and may be placed at the target site to deliver stimulation therapy to a patient. In particular, electrodes 29 on stimulation lead 22 may provide therapy by stimulating the target site.

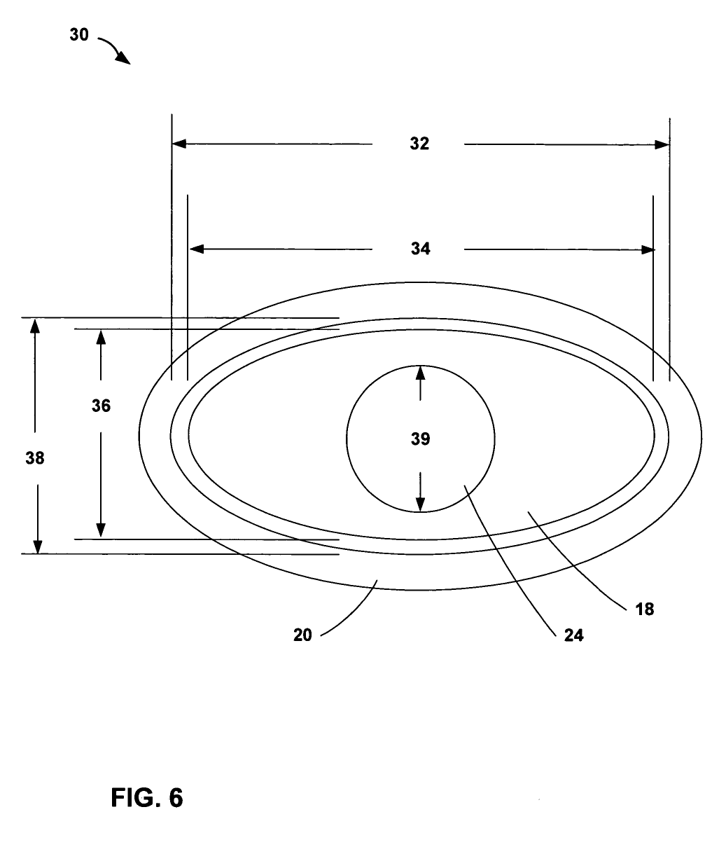

FIG. 6 is a cross-sectional diagram of a stimulation lead introducer 30, including a dilator 18 and an elongated sheath 20 that may be used for percutaneously implanting a stimulation lead. Dilator 18, which may fit inside sheath 20, has a proximal end with a greater circumference than that of the distal end of the dilator. The distal end of dilator 18 includes opening 24, which allows dilator 18 to fit around guidewire 16. In the example of FIG. 6, the cross-section of opening 24 and the dilator proximal end may have a circular or oblong shape. In addition, the cross-section of sheath lumen may include a circular or oblong shape

As shown in FIG. 6, the proximal end of dilator 18 may have an oblong cross-section with a width 34 and a height 36. In one embodiment, width 34 may be approximately 0.175 inches and height 36 may be approximately 0.08 inches. Hence, width 34 is greater than approximately two times height 36. In addition, the distal end of dilator 18 has a height 39, which may be approximately 0.05 inches. In some embodiments, the cross-section of the dilator is circular. In particular, the width of the distal end of dilator 18 may be approximately equal to the height 39 of the distal end of dilator 18. In one embodiment, the cross-section of the sheath lumen has a width 32 of approximately 0.18 inches and a height 38 of approximately 0.085 inches.

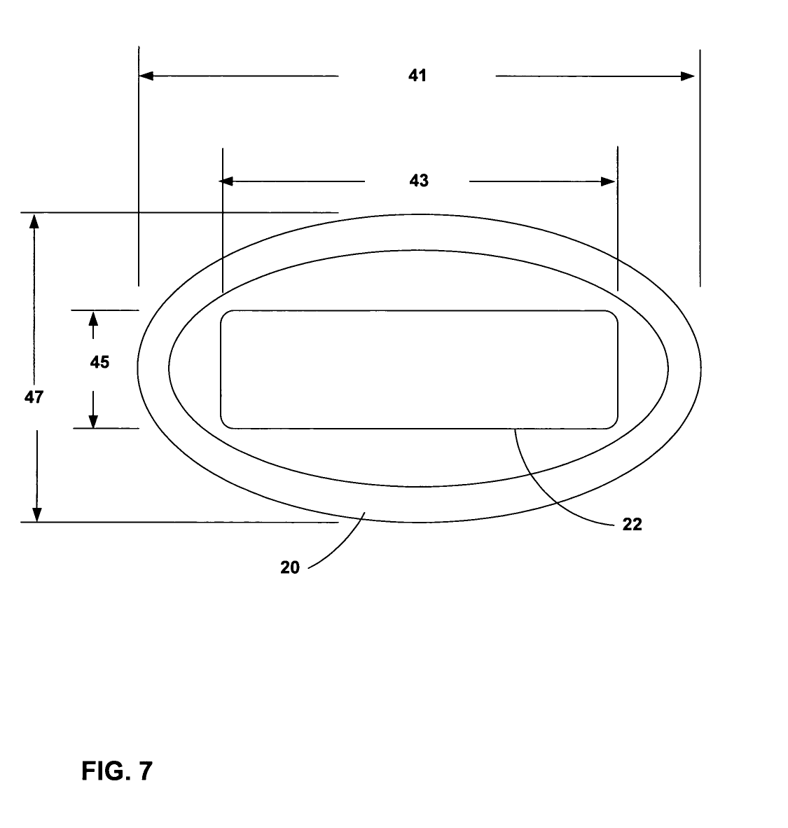

FIG. 7 is a cross-sectional diagram of a stimulation lead 22 passed through a sheath 20 following withdrawal of dilator 18. The outer cross-section of sheath 20 may have a circular or oblong shape. In some cases, stimulation lead 22 may have a substantially rectangular cross-section, as shown in FIG. 7, or an oblong cross-section, providing a generally flat, paddle-like shape. The cross-section of stimulation lead 22 may have a width 43 of approximately 0.15 inches and a height 45 of approximately 0.04 inches. In addition, the outside of sheath 20 may have a width 41 of approximately 0.21 inches and a height 47 of approximately 0.115 inches.

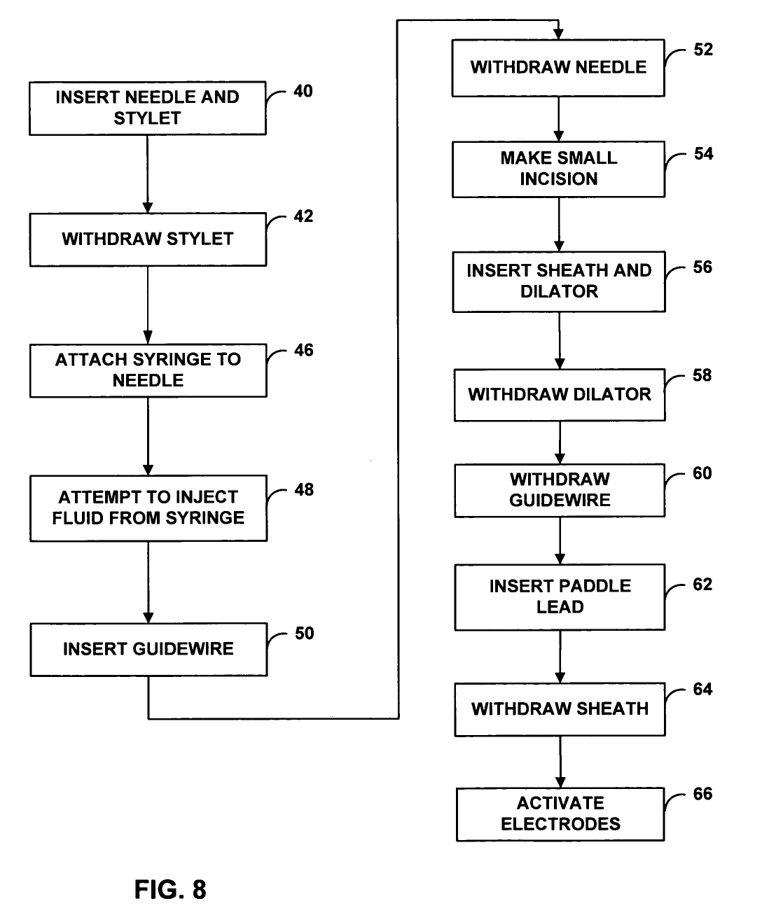

FIG. 8 is a flow diagram illustrating an exemplary technique for percutaneously implanting a stimulation lead by using a stimulation lead introducer 30 as described herein. Initially, a needle assembly is inserted into the epidural region of a patient (40). The needle assembly includes stylet 14 fitted into a lumen defined by needle 12. The lumen may have a diameter between 14 and 18 gauge to allow needle 12 to receive stylet 14. Stylet 14 may fill the lumen of needle 12, preventing tissue coring. In some instances, needle 12 may include a modified Tuohy needle, which has an opening that is angled 45 degrees so that an instrument passing through the needle exits at an angle.

After the needle has been properly inserted into the epidural region of a patient, stylet 14 may be withdrawn (42) from needle 12. In one embodiment, a syringe may be used for placing the needle. If a syringe is used, the syringe is attached to needle 12 (46). Using the syringe may confirm that the needle has been properly placed into the epidural region. In particular, the syringe may attempt to inject fluid, such as air, into the epidural region (48). The fluid from the syringe will encounter substantial resistance if the needle is not correctly placed in the epidural region. On the other hand, a lack of substantial resistance to fluid from the syringe may indicate that the needle 12 has been correctly placed in the epidural region. Once needle 12 has been correctly placed, the syringe may be removed.

In any event, needle 12 is placed in the epidural region, regardless of the presence of the syringe. Upon proper placement of needle 12, a guidewire 16 is inserted (50) into the epidural region via the lumen defined by needle 12. In particular, guidewire 16 slides through the lumen defined by needle 12. Guidewire 16 may be maneuvered through the epidural region until it reaches a target site, wherein the target site is the location where a stimulation lead will be placed for providing stimulation therapy to the patient. In one embodiment, an imaging technique may aid the maneuvering of guidewire 16. For example, the imaging technique may include fluoroscopic imaging.

After the guidewire 16 has entered the body of a patient, the needle is withdrawn (52). In one embodiment, the needle is withdrawn after guidewire 16 has reached the target site for therapy. A small incision may be made (54) proximate the spine to allow entry of a stimulation lead introducer 30. A sheath 20 and a dilator 18, collectively the stimulation lead introducer 30, are inserted (56) through the incision. The dilator 18 is disposed coaxially within sheath 20. At least a portion of stimulation lead introducer 30 may have a cross-section that is substantially oblong.

Upon insertion, stimulation lead introducer 30 is advanced over guidewire 16 until it reaches the therapy target site. As described above, the dilator serves to widen a path surrounding guidewire 16 so a stimulation lead can fit through the path. Sheath 20 serves to maintain the path, which may be oblong, that dilator 18 widens. Once the stimulation lead introducer 30 has reached the therapy target site, dilator 18 is withdrawn from sheath 20 (58). At this point, guidewire 16 is withdrawn (60) from sheath 20, leaving a void within sheath 20. The void is filled by a stimulation lead 22. Stimulation lead 22 is inserted through the incision (62) and advances through sheath 20 until it reaches the therapy target site. Once stimulation lead 22 reaches the therapy target site, sheath 20 is withdrawn (64) from the epidural region. Electrodes 29 on stimulation lead 22 are activated (66) to provide therapy to the patient, e.g., by coupling a proximal end of stimulation lead 22 to a neurostimulator. In one embodiment, a lead extension may be provided to couple stimulation lead 22 to the neurostimulator.

Various embodiments of the invention have been described. These and other embodiments are within the scope of the following claims.

1. A stimulation lead introducer comprising:
an elongated dilator defining a dilator lumen sized to advance over a guidewire, the dilator having a substantially conical distal tip, wherein at least a portion of the conical distal tip has a substantially oblong cross-section; and
an elongated sheath defining a sheath lumen sized to accommodate the dilator or the stimulation lead.

2. The stimulation lead introducer of claim 1, wherein the sheath has a substantially oblong cross-section.

3. The stimulation lead introducer of claim 1, wherein the sheath has a substantially oblong cross-section with a width of the cross-section of the sheath that is greater than approximately two times a height of the cross-section of the sheath.

4. The stimulation lead introducer of claim 1, wherein the dilator lumen has a substantially oblong cross-section.

5. The stimulation lead introducer of claim 1, wherein the sheath lumen has a substantially oblong cross-section.

6. The stimulation lead introducer of claim 1, wherein the sheath comprises a material that is substantially deformable.

7. The stimulation lead introducer of claim 6, wherein the material is polyethylene.

8. The stimulation lead introducer of claim 1, wherein the dilator comprises a material that is substantially deformable.

9. The stimulation lead introducer of claim 8, wherein the material is polyethylene.

10. The stimulation lead introducer of claim 1, wherein the dilator is at least as long as the sheath.

11. The stimulation lead introducer of claim 1, wherein the substantially conical distal tip comprises a proximal opening and a distal opening, the proximal opening having a substantially oblong cross-section and the distal opening having a substantially circular cross-section.

12. The stimulation lead introducer of claim 1, wherein the substantially conical distal tip comprises a proximal opening having an oblong cross-section such that a width of the proximal opening is greater than a height of the proximal opening.

13. The stimulation lead introducer of claim 12, wherein the width of the proximal opening is greater than or equal to approximately three times the height of the proximal opening.

14. The stimulation lead introducer of claim 1, wherein the sheath includes radiopaque material that is viewable under fluoroscopic imaging.

15. The stimulation lead introducer of claim 1, wherein the sheath lumen has a cross-section with a width of the cross-section of the sheath lumen that is greater than approximately two times a height of the cross-section of the sheath lumen.

16. A method for introducing a stimulation lead comprising:
inserting a stimulation lead introducer into an epidural region proximate a spine of a patient via a guidewire, wherein the introducer includes:
an elongated dilator defining a dilator lumen sized to advance over the guidewire, the dilator having a substantially conical distal tip, wherein at least a portion of the conical distal tip has a substantially oblong cross-section, and
an elongated sheath defining a sheath lumen sized to accommodate the dilator or the stimulation lead;
withdrawing the dilator from the sheath; and
introducing a stimulation lead to a target site within the epidural region via the sheath.

17. The method of claim 16, further comprising:
inserting a needle with a stylet into the epidural region proximate a spine of a patient;
withdrawing the stylet from the needle;
inserting the guidewire into the needle such that a distal end of the guidewire extends to the target site within the epidural region;
withdrawing the needle;
inserting the stimulation lead introducer into the patient via the guidewire following withdrawal of the needle;
withdrawing the guidewire; and
introducing the stimulation lead via the sheath following withdrawal of the dilator and the guidewire.

18. The method of claim 17, further comprising withdrawing the sheath.

19. The method of claim 17, further comprising activating the stimulation lead to stimulate a nerve.

20. The method of claim 17, further comprising attaching a syringe to the needle, prior to inserting the guidewire into the needle, and attempting to inject fluid into the epidural region via the syringe and the needle to evaluate a position of the needle.

21. The method of claim 17, further comprising using an imaging technique to visualize introduction of the stimulation lead.

22. The method of claim 21, wherein the imaging technique comprises fluoroscopic imaging.

23. The method of claim 17, wherein the needle is a Tuohy needle.

24. The method of claim 16, wherein the sheath has a substantially oblong cross-section.

25. The method of claim 16, wherein the sheath has a substantially oblong cross-section with a width of the cross-section of the sheath that is greater than approximately two times a height of the cross-section of the sheath.

26. The method of claim 16, wherein the dilator lumen has a substantially oblong cross-section.

27. The method of claim 16, wherein the sheath lumen has a substantially oblong cross-section.

28. The method of claim 16, wherein the sheath comprises a material that is substantially deformable.

29. The method of claim 28, wherein the material is polyethylene.

30. The method of claim 16, wherein the dilator comprises a material that is substantially deformable.

31. The method of claim 30, wherein the material is polyethylene.

32. The method of claim 16, wherein the dilator is at least as long as the sheath.

33. The method of claim 16, wherein the substantially conical distal tip comprises a proximal opening and a distal opening, the proximal opening having a substantially oblong cross-section and the distal opening having a substantially circular cross-section.

34. The method of claim 16, wherein the substantially conical distal tip comprises a proximal opening having an oblong cross-section such that a width of the proximal opening is greater than a height of the proximal opening.

35. The method of claim 34, wherein the width of the proximal opening is greater than or equal to approximately three times the height of the proximal opening.

36. The method of claim 16, wherein the sheath includes radiopaque material that is viewable under fluoroscopic imaging.

37. The method of claim 16, wherein the sheath lumen has a cross-section with a width of the cross-section of the sheath lumen that is greater than approximately two times a height of the cross-section of the sheath lumen.

38. A dilator for widening a path for a stimulation lead to travel through an epidural region proximate a spine of a patient, the dilator having a proximal end and a distal end, wherein the dilator defines a dilator lumen sized to advance over a guidewire, the dilator having a substantially conical distal tip, wherein at least a portion of the conical distal tip has a substantially oblong cross-section.

39. The dilator of claim 38, wherein the dilator is formed from a material that is substantially deformable.

40. The dilator of claim 39, wherein the material is polyethylene.

41. The dilator of claim 38, wherein the dilator lumen has a substantially oblong cross-section.

42. The dilator of claim 35, wherein the substantially conical distal tip comprises a proximal opening and a distal opening, the proximal opening having a substantially oblong cross-section and the distal opening having a substantially circular cross-section.

43. The dilator of claim 42, wherein the width of the proximal opening is greater than or equal to approximately three times the height of the proximal opening.
